(19) United States
(12) Patent Application Publication
(10) Pub. No.: US 2016/0374851 A1
(43) Pub. Date: Dec. 29, 2016

(54) FAUX-STONE APPARATUS AND PROCESSES OF USING THE SAME FOR PERSONAL TREATMENT

(71) Applicant: Heidi L. Smith, Atlanta, GA (US)

(72) Inventor: Heidi L. Smith, Atlanta, GA (US)

(21) Appl. No.: 15/190,602

(22) Filed: Jun. 23, 2016

Related U.S. Application Data
(60) Provisional application No. 62/183,382, filed on Jun. 23, 2015.

(51) Int. Cl. A61F 7/02; A61H 7/00

(52) U.S. Cl. CPC A61F 7/02; A61H 7/001; A61F 2007/0207; A61F 2007/0223; A61F 2007/0279; A61F 2007/0266; A61F 2007/0088

(57) ABSTRACT

A thermal-treatment process including providing at least one faux-stone apparatus, such as in a pre-packaged kit. The process in some implementations includes providing a faux-stone usage instruction, such as on kit packaging or with the faux-stone apparatus(es) within the packaging. The process includes heating or cooling the faux-stone apparatus, yielding a thermally-effected faux-stone apparatus. The process also includes applying at least one thermally-effected faux-stone apparatus to a user. Each apparatus includes a natural granular filler comprising a multiplicity of natural granules configured to (i) retain heat received by conduction or radiation and by radiation and/or conduction transfer the heat to a user and (ii) be cooled and receive heat from the user body. Each apparatus includes a natural cover sized and shaped to fit in its entirety within a user hand, is generally round, oval or irregularly shaped to look like a real stone, and encases the granular filler.

### TECHNICAL FIELD

This disclosure relates generally to body treatment implements and processes and, more particularly, to faux-stone apparatus for thermal and/or pressure treatment of a user's body.

### BACKGROUND

This section provides background information related to the present disclosure which is not necessarily prior art.

Many of the benefits of external body treatments, such as those using heat, cold, and pressure points, have been known for centuries.

Modern equivalents of systems like the Roman and Greek hot and cold baths and steam rooms, such as saunas and whirlpools, are expensive to install and maintain. Their large size also deters from more-common usage. And often full body treatment would not be most effective for desired purposes.

Localized application of heat, cold, or pressure to the body has been found to foster special relaxing and healing benefits. Use of heated rocks or stones has been reported as far back as two-thousand years ago in regions such as China, India, Europe, Africa, and South America. Natural stones can be relatively expensive, being marble, granite, or jasper, for instance. While weight of natural stones can promote desired effects, the weight can also limit portability, such as in a user's purse or carry bag.

Modern products providing localized body treatments are relatively complicated and expensive. Many are electric, requiring batteries or power cords. Products include vibration devices, electric heat pads, and those providing infrared heat therapy.

Many products contain expensive gels or other complex chemical combinations. These products are also not ecofriendly or sustainable. They typically require multiple man-made materials, for instance, such as plastic casings, electrical components, and/or and synthetic chemicals.

### SUMMARY

There is a need for convenient systems and processes or methods for providing heat, cold, and pressure treatment. The faux-stone apparatus of the present disclosure are portable, cost-effective, and configured to provide thermal and pressure treatment. One or more stones can be transported and used, such stones from a pre-assembled faux-stone kit, assembly, or system.

The present technology includes in one aspect, a thermal-treatment process including providing at least one faux-stone apparatus, such as in a pre-packaged kit. The process in some implementations includes providing a faux-stone usage instructions. The instructions may be provided on kit packaging, for instance, or with the faux-stone apparatus within the packaging.

The process includes heating or cooling the faux-stone apparatus, yielding a thermally-effected faux-stone apparatus.

The process also includes applying at least one thermally-effected faux-stone apparatus to a user.

Each faux-stone apparatus includes a filler, such as a granular filler and, more particularly such as a natural granular filler.

The filler in some implementations includes multiple natural granules configured to (i) retain heat received by conduction or radiation and by radiation and/or conduction transfer the heat to a user and/or (ii) be cooled and receive heat from the user body.

In various embodiments, each faux-stone apparatus includes a natural cover sized and shaped to fit in its entirety within a user hand.

The cover of each faux-stone apparatus encases the granular filler and is in some implementations generally round, oval, or irregularly shaped, to look like a real stone.

The encapsulating cover in various embodiments includes a natural and soft material such as cotton holding a natural filler such as buckwheat, rice, millet, or flaxseed. Other example fillers are provided below.

In one embodiment, the cover of the faux-stone includes an easily cleanable surface, such as that which can be cleaned simply by wiping. In some embodiments, the cover is machine washable.

In various embodiments, such as those in which the cover is not machine washable, the faux-stone product can be provided with, or sold separately from, one or more sleeves or coverlets, each configured to encapsulate at least one faux-stone. Each coverlet can be machine washable, for instance.

The faux-stone apparatus are applied to the body for various purposes after the stone(s) are heated, cooled, or at room temperature. Heating methods include but are not limited to heating in an upper compartment of a double boiler system, providing heat to the compartment by steam rising from boiling water. Cooling methods include but are not limited to cooling the apparatus using ice or a refrigerator.

Whether heated, cooled, or ambient (e.g., room temperature), a faux stone can be applied to any external part of the body, such as head, neck, chest, arms, hands, back, legs, and feet. In some implementations, faux stones are placed simultaneously against more than one part of the body.

In an implementation, the faux-stone apparatus is provided to users in a kit including instructions for use. The instructions describe manners of heating and cooling the faux stones. The instructions can also refer to most-effective or recommended body locations at which to apply the faux-stone apparatus. The recommended locations can include one or more body points identified in external body treatment schemes, or energy system. Such example schemes or energy systems include, but are not limited to, a chakra system, an acupuncture system—e.g., a 400 point system or any subset thereof, and a meridian system—e.g., 12 standard meridian points or any subset thereof.

The faux-stone apparatus is in various embodiments packaged for display, sale, storage, and transport.

Their relatively light weight and soft exterior make them easy and pleasant to hold and use.

Their ability to be easily heated and cooled facilitates thermal uses, and the natural materials used in various embodiments are sustainable and eco-friendly.

### DESCRIPTION OF THE DRAWINGS

FIG. 1A illustrates a stack of example faux-stone bags, pouches or apparatus, for hot, cold, and/or pressure treatment.

FIG. 1B illustrates a schematic side view of one of the faux-stone apparatus.

FIG. 1C shows the example faux-stone apparatus having two irregular shaped pieces of material sewn together to create a pouch filled with a filler.

FIG. 1D illustrates example dimensions of a faux-stone apparatus. In various implementations, any of the dimensions are smaller or larger.

FIGS. 2A-2C illustrate example storage/display environments.

FIGS. 3A-3E illustrate example storage/display kits or packages for a plurality of faux-stone apparatus.

FIG. 4 shows schematically a front and side view of a human body highlighted with example application points at which to apply the faux stone apparatus of the present technology.

FIG. 5-18 show views of various embodiments of faux-stone apparatus, including those showing different stone finishes, those with marked (and scented) messages, and those with a storage/display environment, kit or package.

FIGS. 19 and 20 show three faux stone apparatus positioned on a user's back.

FIGS. 21-23 show schematically example coverlets positioned about a faux-stone apparatus according to the present technology, wherein FIG. 21 shows a see-through view of the coverlet in order to show the faux-stone inside, FIG. 22 shows a side view of the combination with the slit in the coverlet pulled open to expose the faux stone inside, and FIG. 23 shows a side view of the combination of FIGS. 21 and 22.

The figures are not necessarily to scale and some features may be exaggerated or minimized, such as to show details of particular components. In some instances, well-known components, systems, materials or methods have not been described in detail in order to avoid obscuring the present disclosure.

Specific structural and functional details disclosed herein are not to be interpreted as limiting, but merely as a basis for the claims and as a representative basis for teaching one skilled in the art to variously employ the present disclosure.

### DETAILED DESCRIPTION

There is a need for the faux-stone apparatus described herein for applying heat, cold, and/or pressure stimuli selectively to one or more parts of a user body.

The faux stones enable effective treatments when placed at preferred body locations when the stones are hot or cold.

The stones are in various implementations kept at room temperature for effective treatments as well, such as to apply pressure to the user body with the ambient-temperature faux-stone apparatus. Pressure applied via the faux-stone apparatus is generated by mass/gravity of the apparatus and/or pressure by an applicator such as a therapist or the user.

Novel pressure treatments can also be effected using heated or cooled faux-stone apparatus.

While select embodiments of the faux-stone apparatus are described in connection with human use, the apparatus can be used with most animals.

I. Faux-Stone Apparatus—General Overview

The faux stones described herein can be referred to by any of a wide variety of terms. Each faux stone, or collection of two or more faux stones, can be referred to, for instance, as a system, apparatus, instrument, implement, pouch, bag, tool, any of these preceded by stone or faux-stone, or imitation-stone (e.g., faux-stone pouch, imitation-stone apparatus), natural faux stone, natural-looking faux stone, similar, or other.

The faux-stone apparatus are referred to primarily as rocks or stones here because they are made—e.g., sized, shaped, colored, and including material—to look like real, natural stones or rocks. In some implementations, the shape of the faux-stone apparatus resembles river rock stone.

Each faux-stone apparatus, or faux-stone, has a cover. The cover is in some embodiments a natural-material cover. The cover is referred to primarily herein as a natural cover but is not limited to being natural. It can consist of a man-made, or synthetic, material, for instance.

In some implementations the system includes multiple faux-stones apparatus provided together. The faux-stone apparatus can have generally the same or different sizes. In one embodiment, the system is provided with at least one faux-stone being smaller (e.g., a miniature version) than another of the faux stones.

In a contemplated embodiment, three or more of the faux stones have respective differing sizes so that each is larger or smaller than at least one of the other faux stones. The stones in these cases can be stacked, for instance, with a first, largest diameter or width stone on the bottom, a next largest (smaller than the first) placed atop of the first, and a third, still smaller one atop of the second. There may be four, five or more stones sized and stackable in this way.

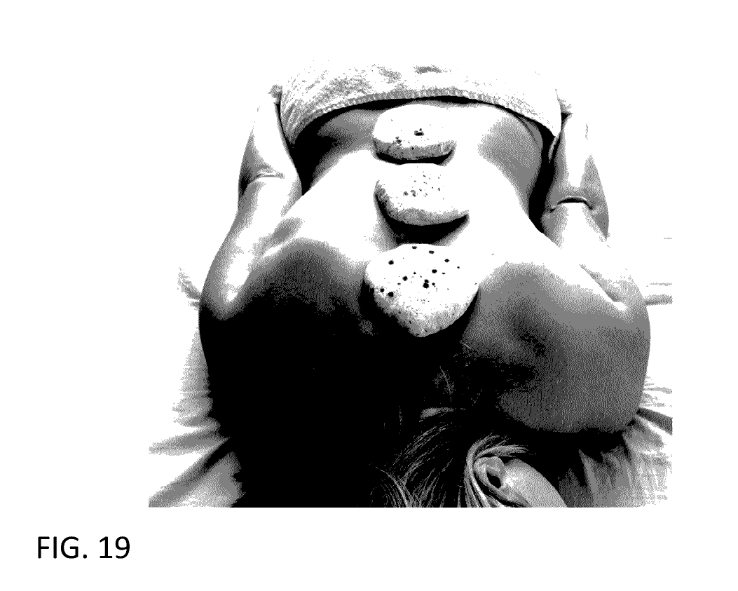

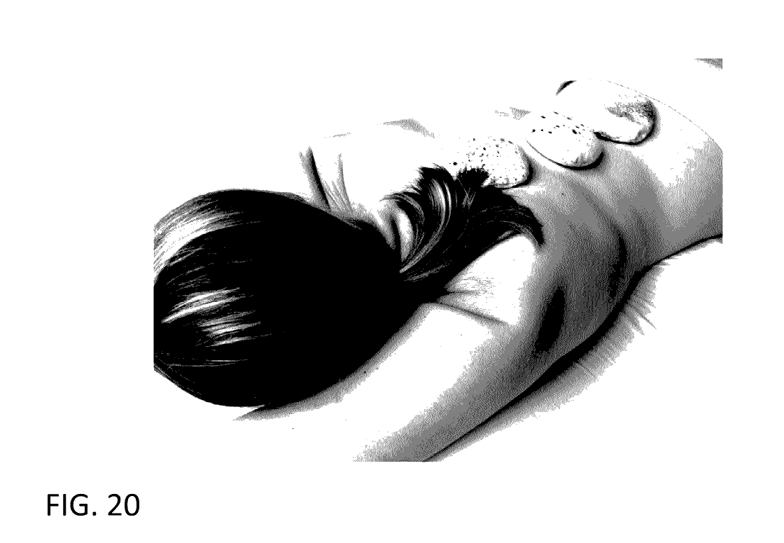

In a contemplated embodiment, the different-sized stones are provided in a kit or package and the different-sized stones can be used to differing effects on different parts of the user. Further contemplated, instructions provided with the kit can describe where a smaller faux stone is recommended to be used and where a larger one is to be used—such as a smaller one being placed on a user wrist and a larger one being placed on a larger body part such as on the user back—reference FIGS. 19 and 20 showing use on a user back and FIG. 4 showing various example faux-stone application locations on a user body.

In some implementations, a faux-stone apparatus includes two irregular shaped pieces of material sewn together to create a pouch filled with a filler. An example filler is buckwheat, such as an organic buckwheat. Other example fillers are provided below.

The filler is configured to, during operation of the faux-stone apparatus, store and release heat for providing warm or hot heat treatment to a user. The filler is configured to, for instance, receive heat input to the apparatus.

The heat can be received, such as from a double boiler or microwave, by conduction and/or radiation. The filler is further configured to retain heat received and, when the apparatus is positioned adjacent a portion of a user body after being heated, to transfer heat by radiation and/or conduction to the user body to heat the body portion.

The filler is configured to, during operation of the faux-stone apparatus, provide cooling or cold treatment to the user. The filler is configured, for instance, to, during operation of the faux-stone apparatus, cool when exposed to a lower-temperature source or environment by transferring heat to the source or environment, and, when the apparatus is positioned adjacent the user body after being cooled, receive heat from the user body, thereby cooling the body portion.

In some implementations, the faux-stone apparatus cover includes cotton. Other example faux-stone-cover materials are described below. In some implementations, the faux-stone apparatus 100 may be scented, such as by the cover and/or filler being scented.

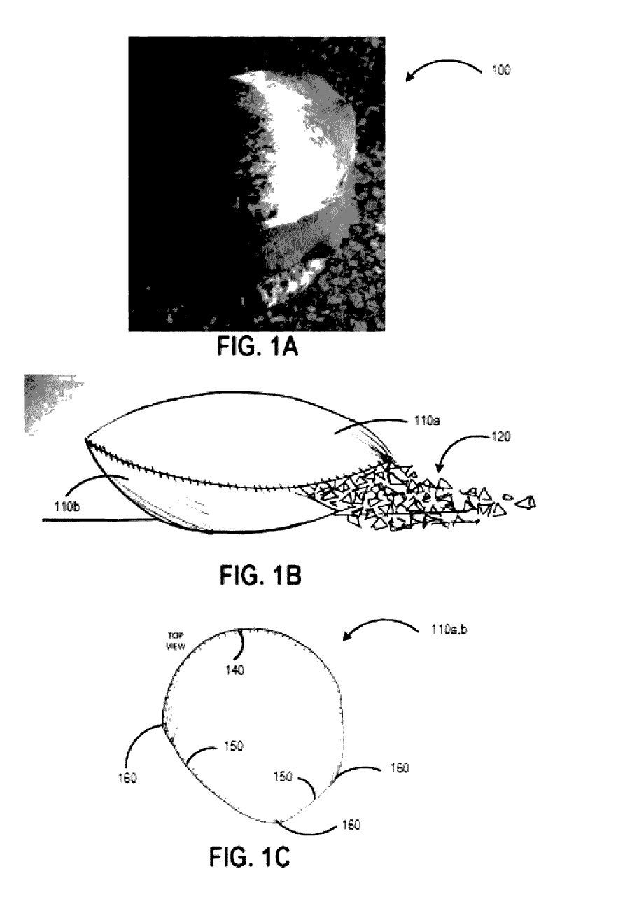

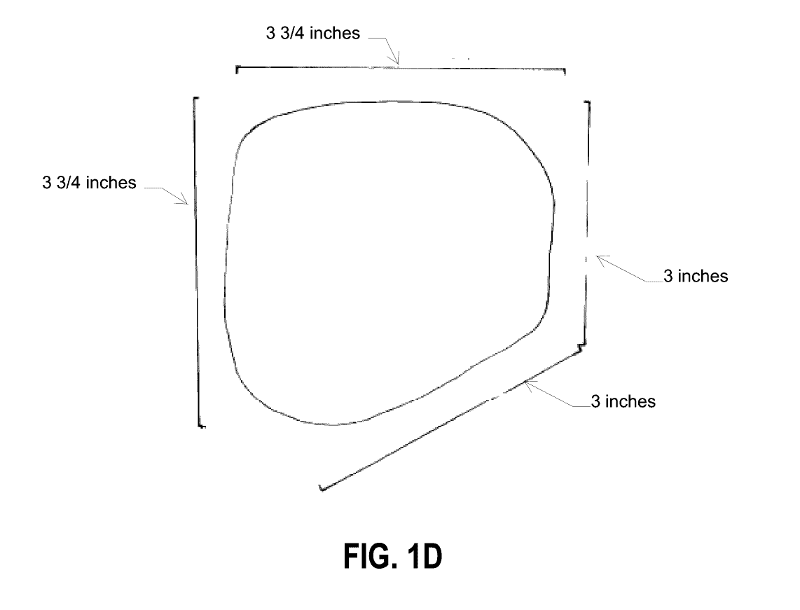

II. Stacked Arrangement, Example Exteriors—FIG. 1A

FIG. 1A illustrates an example implementation of the faux-stone apparatus 100 for hot, cold, and or ambient-temperature treatment. The figure shows a perspective view of multiple faux-stones stacked. The various coloring and patterns of the faux stones in the stack are examples of the authentic, natural stone look that can be applied to the cover such as by dying, to make the apparatus resemble actual rocks or stones—i.e., each natural stone is unique.

In some implementations, the material 110a, b consists of or includes cotton. In some implementations, the material 110a, b may be organic cotton. In some implementations, the material may be a flannel cotton. In some implementations, the material 110a, b may be any other suitable material. In a contemplated embodiment, the material 110a, b includes wool, such as a flannel wool.

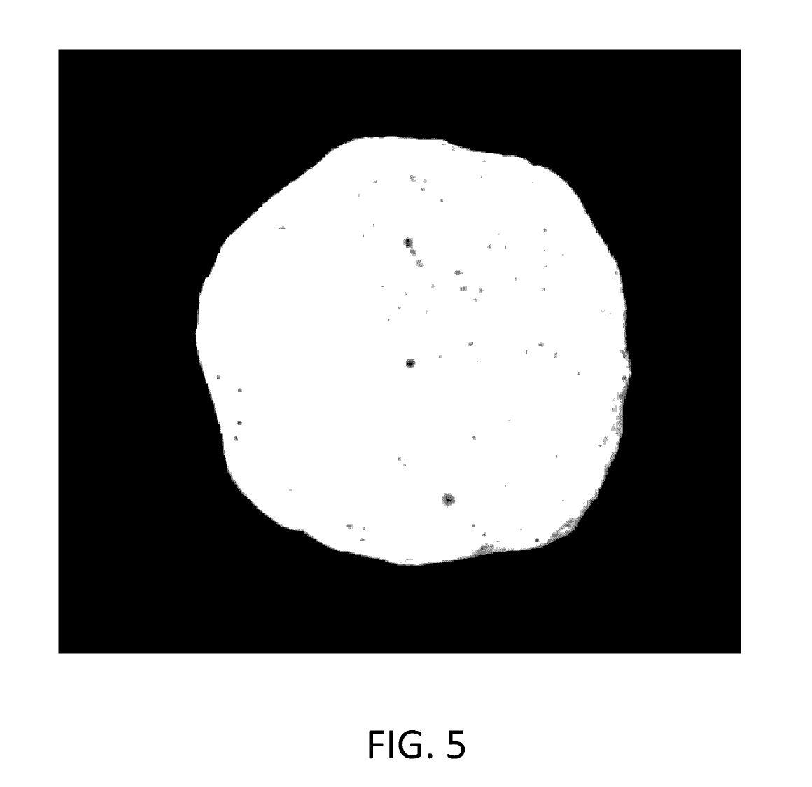

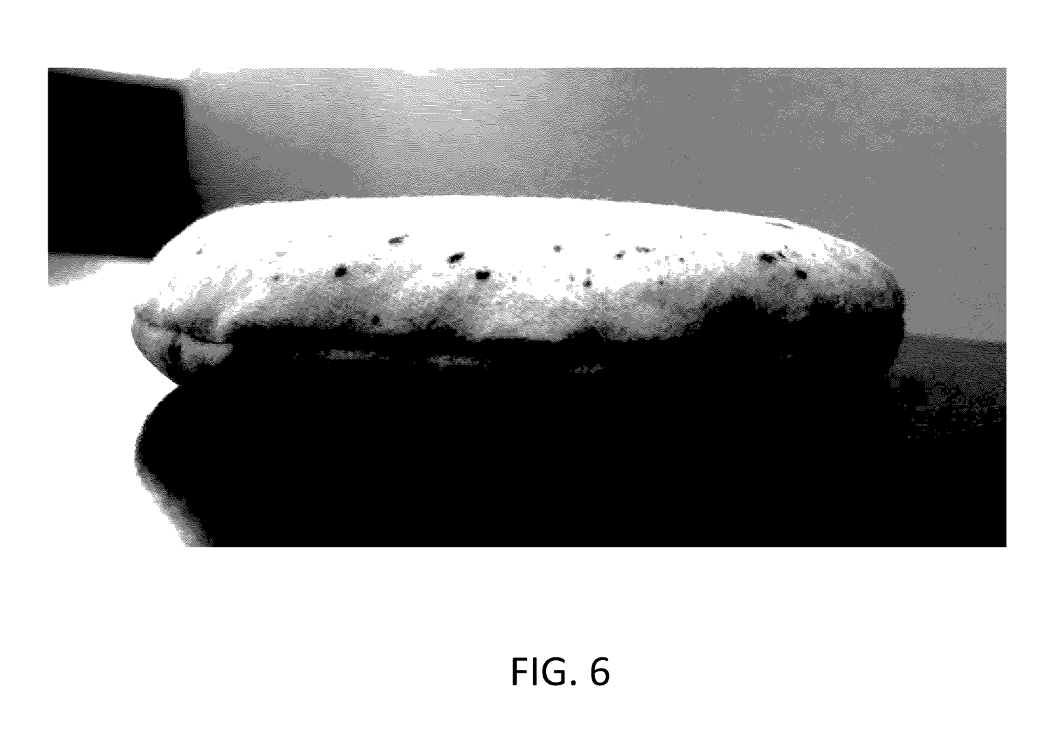

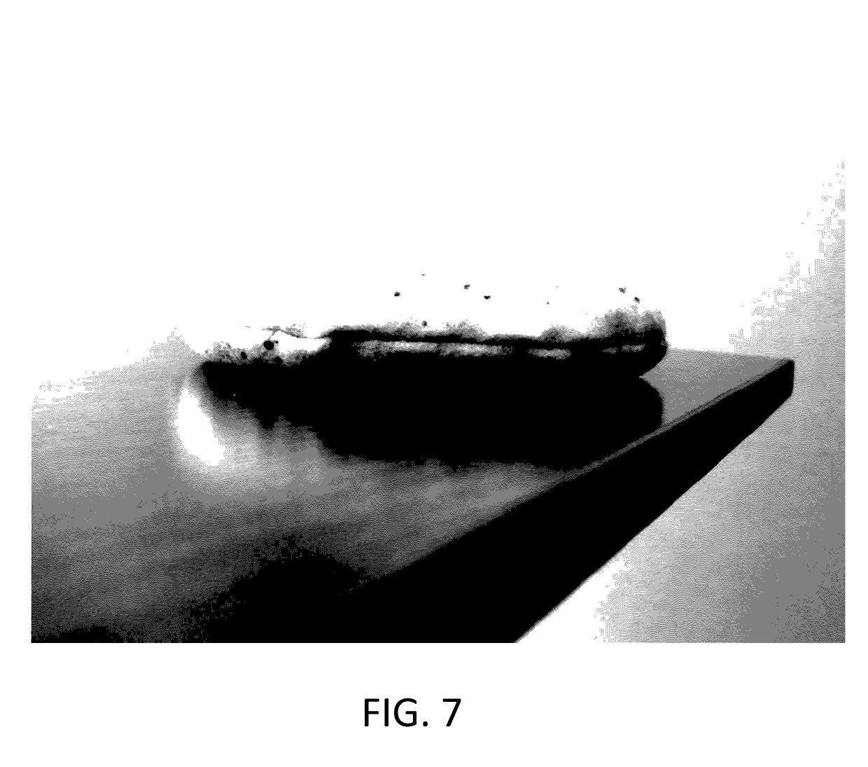

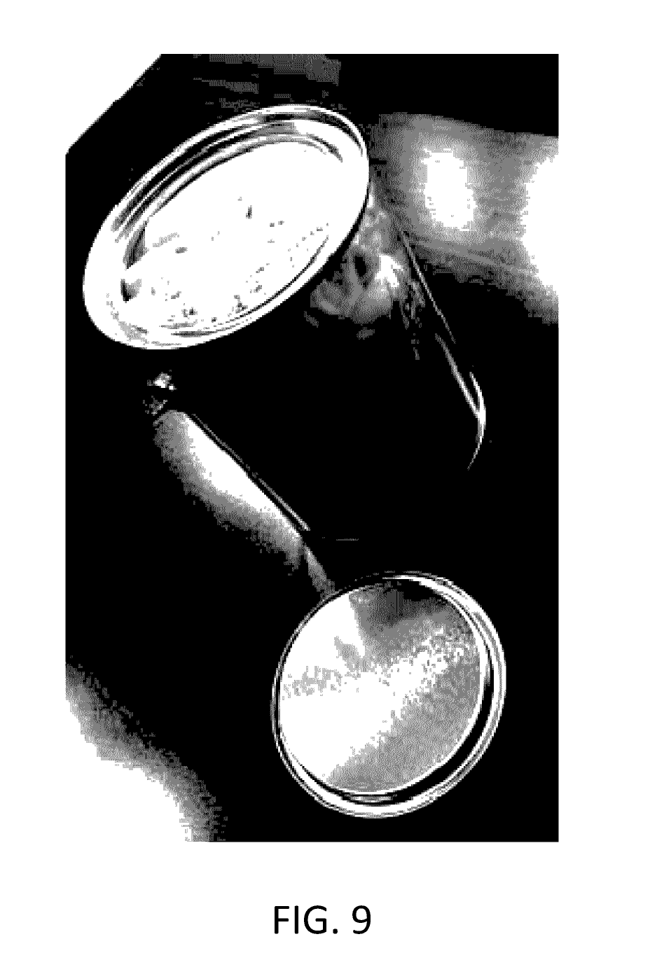

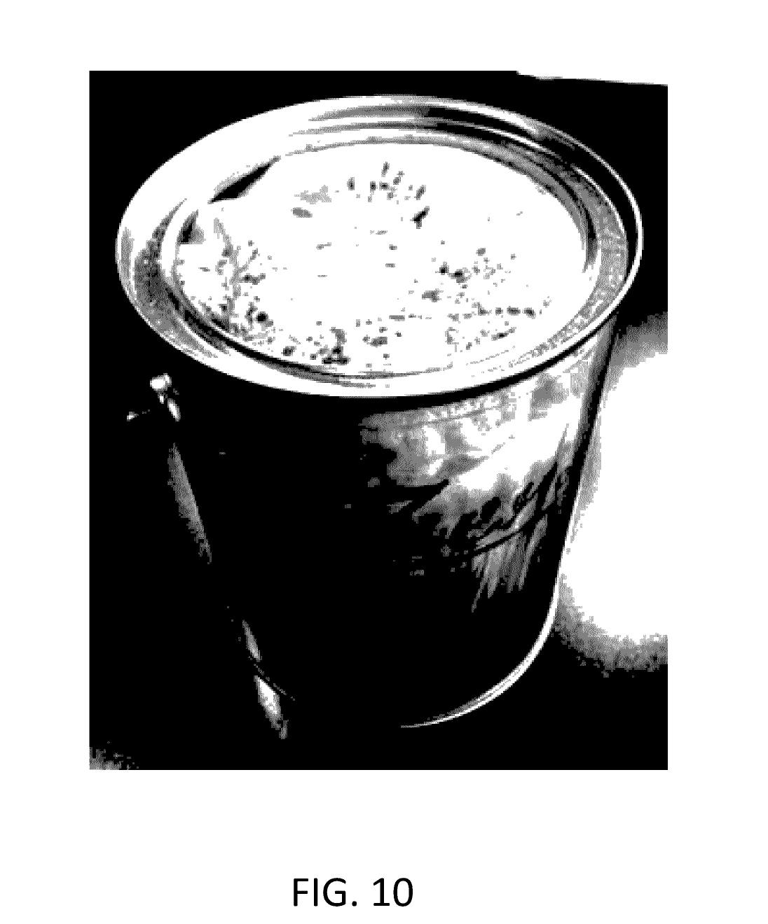

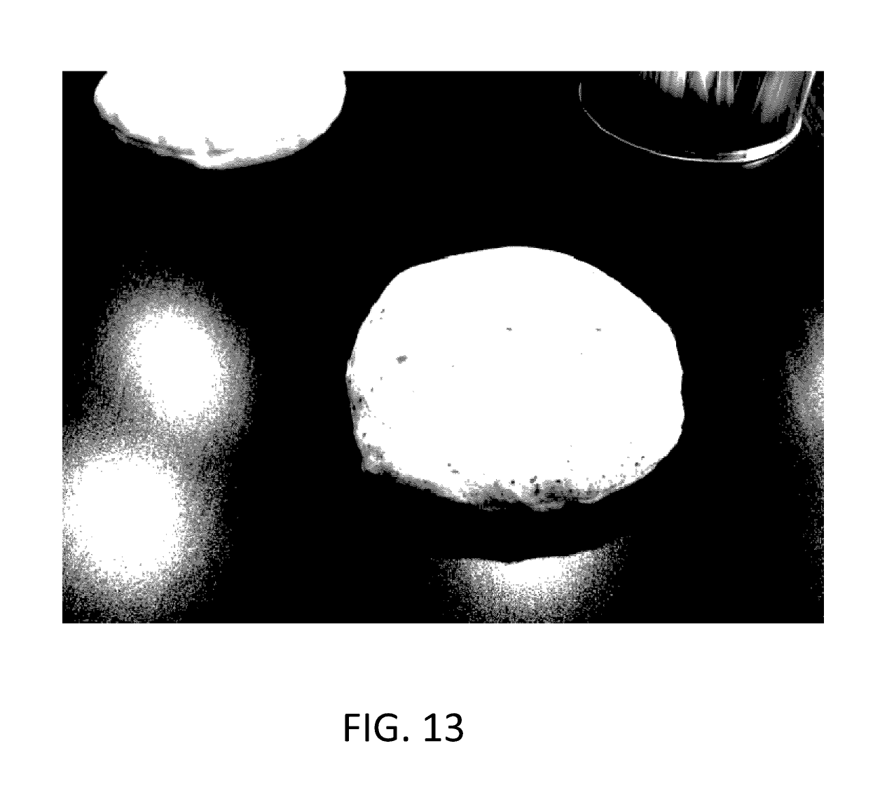

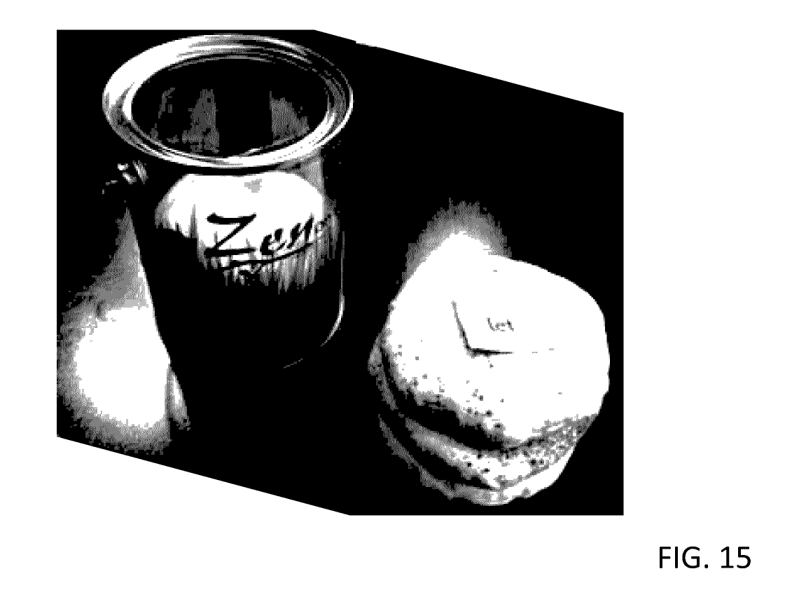

In some implementations, the material 110a, b may be dyed to achieve a desired look. In some implementations, the visual design may be speckled, for instance by the speckling being provided on the material or through a secondary dye process. For examples of speckled finishes, see e.g., FIGS. 5-18.

In some implementations, the design is multi-toned.

In various implementations, the design is solid. In some implementations, the design includes any suitable design, such as by the material 110a, b including any suitable color, such as coloring giving the apparatus a more realistic look to real stone or rock.

The material is in some embodiments colored and/or effected otherwise in a manner corresponding to a theme or season, such as with colors associated with a holiday.

In some implementations, the faux-stone apparatus 100 is scented.

III. Primary Components—FIGS. 1B and 1C

FIG. 1B illustrates a schematic side view, and FIG. 1C a schematic top view, of the faux-stone apparatus 100 showing the primary components.

As shown in FIGS. 1B, 1C, the faux-stone apparatus 100 can include two irregular shaped pieces of cover material 110a, b sewn together to create a pouch filled with a filler 120.

In other cases, the apparatus is more regular, such as being closer to perfect round or oval in top profile, side profile, and/or other view.

In a contemplated embodiment, the cover consists of a single material. The single material can include an opening through which the filler is added during manufacture, and which is closed after the filling such as by sewing.

In some implementations, the filler 120 is a natural filler. A natural filler consists of or includes products of nature, such as plant products.

In some embodiments the filler 120 is or includes preferably an organic filler, such as an organic buckwheat filler. In some implementations, the filler 120 is or includes raw buckwheat groats, and in some cases, organic raw buckwheat groats 120.

In some implementations, the filler 120 includes any of rice, millet, dried corn, beans, flaxseed, cherry pits, or silica beads, or any other suitable filler for the purposes described herein.

In one embodiment, the filler 120 includes pellets.

In one embodiment the filler 120 includes a fluid and the cover is configured to contain the fluid, such as by being generally non-porous. The fluid in some implementations includes a gel. The fluid may be partially or fully natural, and can be partially or completely organic.

In some implementations, each faux-stone apparatus 100 is configured (e.g., sized, shaped, colored) to resemble a rock or stone, such as river rock stone. FIG. 1C illustrates an example implementation of a pattern of the irregular shaped pieces of material 110a, b.

In some implementations, at least a portion 140 of the perimeter of the material 110 is shaped to form part of a circle or oval. In some implementations, at least a portion 150 of the perimeter of the material 110 is generally linear or straight. In some implementations, some or all of the sides include a change of direction 160, which can be referred to in some cases as corners. The changes in direction 160 are rounded to an extent.

In a contemplated embodiment the filler includes a fluid-granule mixture. A fluid, such as a gel, is configured to, during operation of the faux-stone apparatus, store and release heat for providing warm, hot, or heat treatment to a user. The fluid is configured, for instance, to receive heat input to the apparatus. The heat can be received, such as from a double boiler or microwave, by conduction and/or radiation. The fluid is further configured to retain heat received and, when the apparatus is positioned adjacent a portion of a user body after being heated, transfer heat by radiation and conduction to the user body to heat the body portion.

For embodiments in which the filler includes a mixture of a fluid (e.g., gel) and granules, the fluid and/or the granules can be configured with these heating properties.

The fluid (e.g., gel) is configured to, during operation of the faux-stone apparatus, provide cooling or cold treatment to a user. The fluid is configured, for instance, to, during operation of the faux-stone apparatus, cool when exposed to a lower-temperature source or environment by transferring heat to the source or environment, and, when the apparatus is positioned adjacent the user body after being cooled, receive heat from the user body thereby cooling the body portion.

For embodiments in which the filler includes a mixture of a fluid (e.g., gel) and granules, the fluid and/or the granules can be configured with these cooling properties.

IV. Example Dimensions—FIG. 1D

FIG. 1D illustrates example dimensions of an implementation of a faux-stone apparatus according to this disclosure. In some implementations, the dimensions may be smaller or larger.

In some implementations, the shape of the faux-stone apparatus 100 has a generally circular or oval top profile. In some implementations, the shape of the faux-stone apparatus 100 is any other suitable shape, regular or irregular.

In various embodiments, at least one of the faux-stones covers is made by cutting one or more pieces of cover material from a three and three-fourths inch (3¾″) square piece of fabric, or an approximately 2¾″ cut from a square piece of fabric for a smaller, for a smaller (miniature) version of the apparatus. The cutting performed to achieve a somewhat arbitrary diameter for the end faux stone. In some implementations, forming the covers of faux stones is done in such a way that each stone is substantially different from the others, as stones in nature are unique.

In various embodiments, some or every resulting faux-stone apparatus is sized and shaped to fit in its entirety within a hand of the user. The cover is in some cases sized and shaped to fit mostly or entirely in a palm of the hand of the user.

The cover in various embodiments has a maximum width between about two inches and about five inches. The cover in embodiments has a maximum width between about two inches and about four inches, and in others, a maximum width of about between about two inches and about three inches.

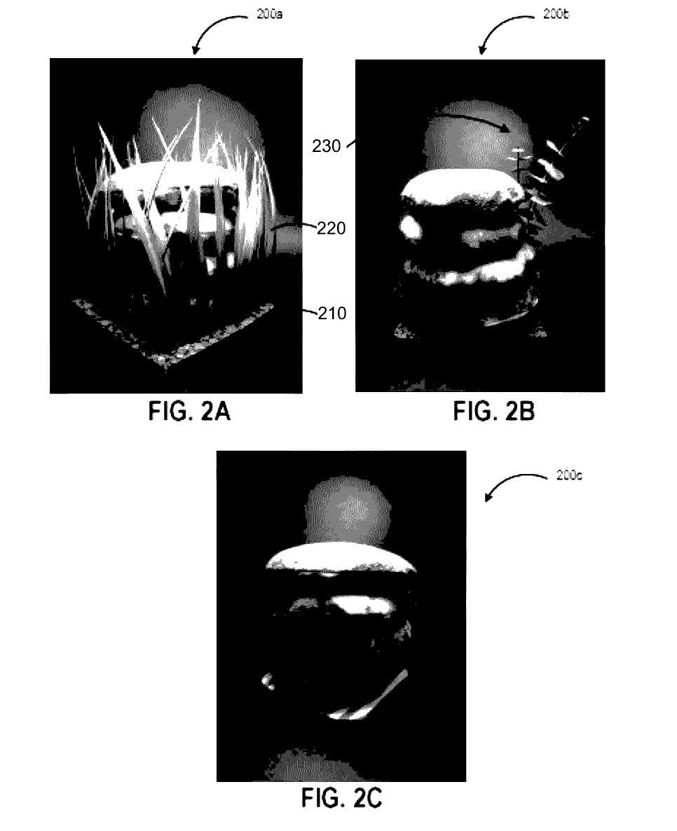

V. First Example Display Arrangement—FIG. 2A-2C

FIGS. 2A-2C illustrate example storage and/or display arrangements or environments 200a, b, c for a plurality of faux-stone apparatus (e.g., apparatus 100). The views further show examples of various coloring that can be provided for the covers to make the apparatus resemble natural stones.

In some implementations, a plurality of faux-stone apparatus according to the present disclosure may be stacked on top of each other and placed on a platform 210.

In some implementations, the platform 210 is square. In some implementations, the platform 210 is any suitable shape. The platform can include or be made to look like a stone, such as marble, or to look like dirt or other natural-environment feature.

In some implementations, the platform 210 may have faux grass 220 extending therefrom or faux plants 230. In some implementations, the faux grass 220 may extend from the platform 210 on each side of the platform 210. In some implementations, the faux grass 220 may be any green elongated material.

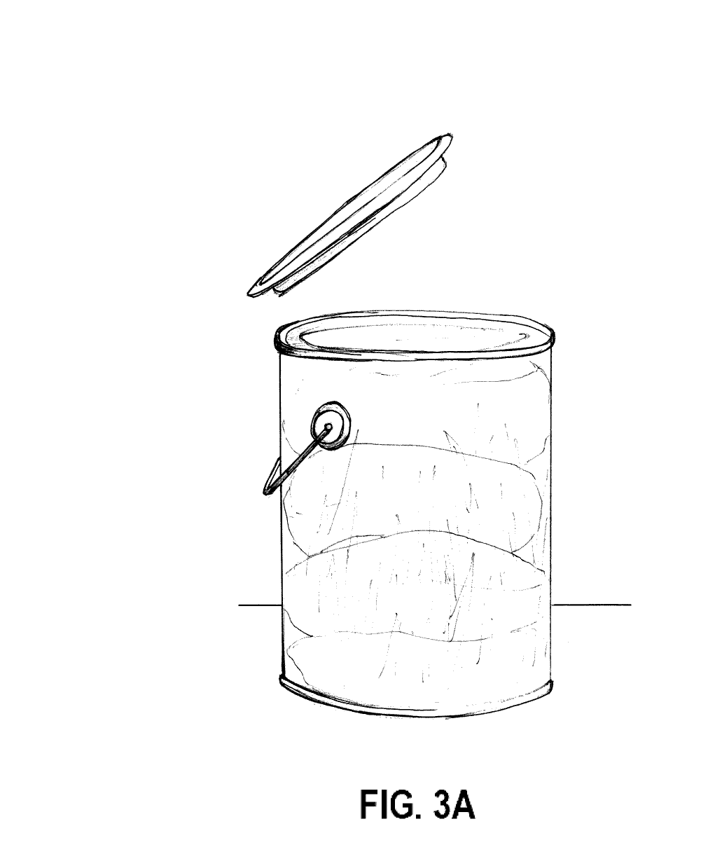

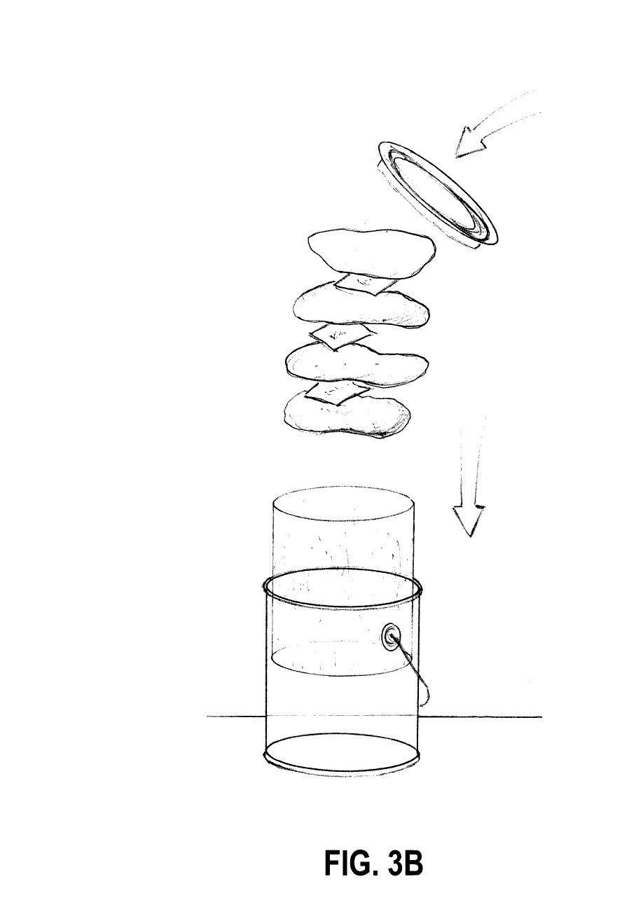

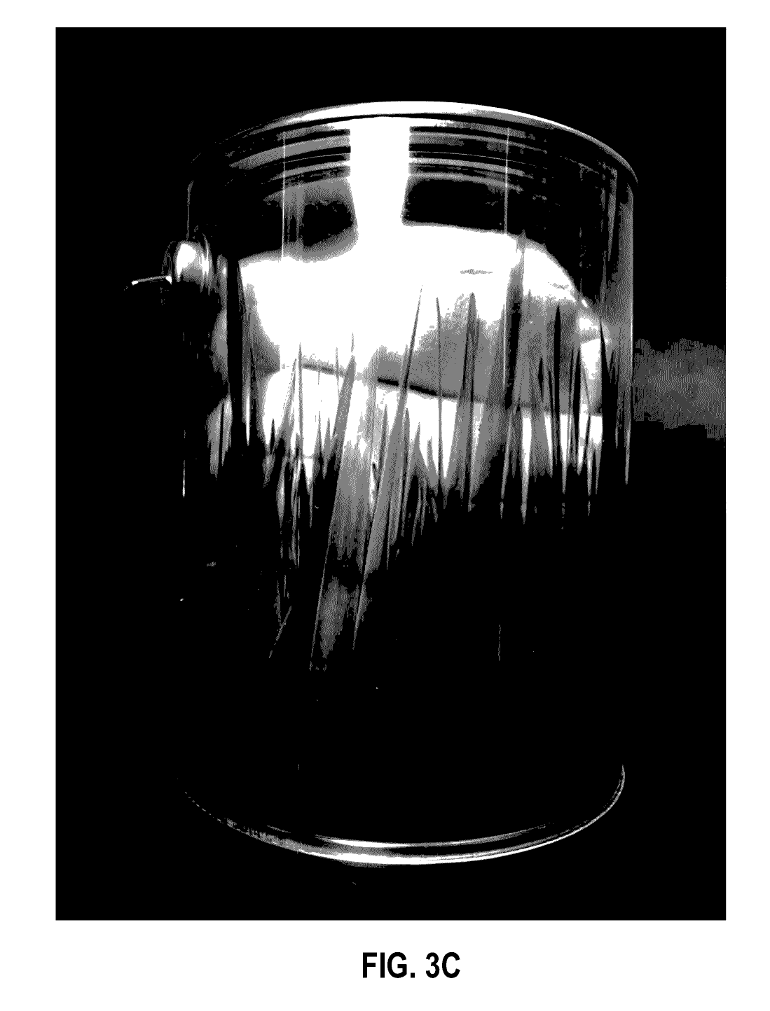

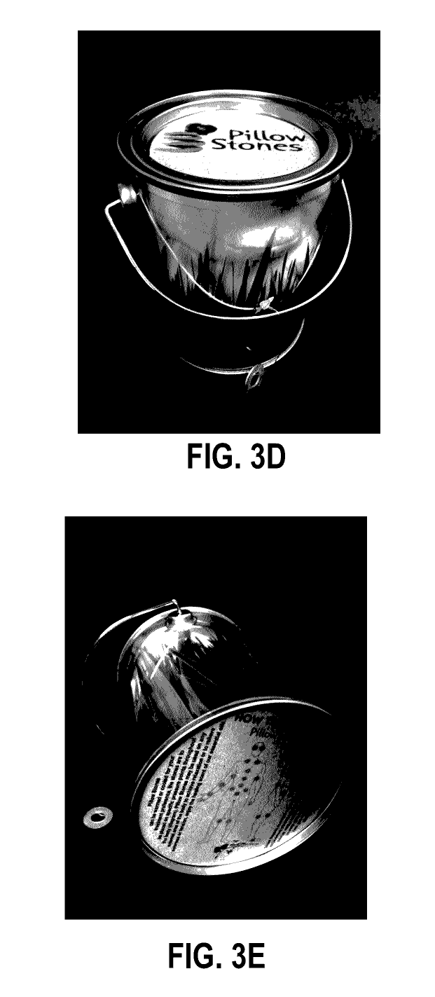

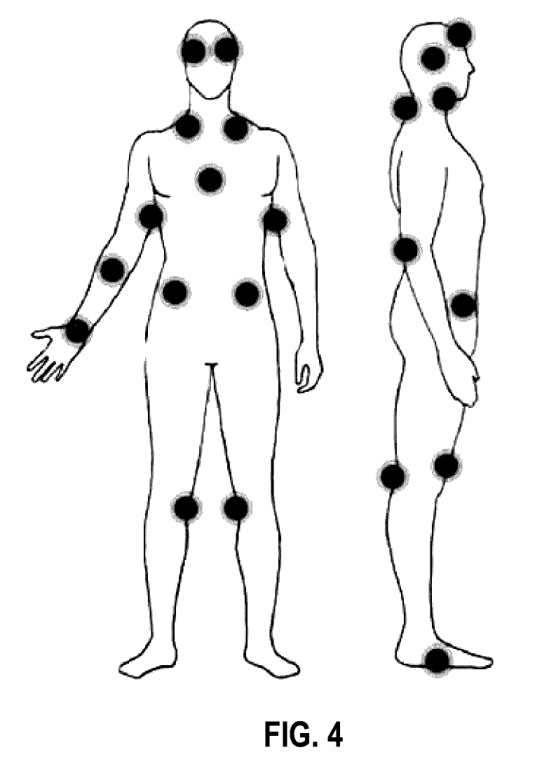

VI. Second Example Display Arrangement—FIG. 3A-3E

FIGS. 3A-3E illustrate another example storage/display environment for a plurality of faux-stone apparatus, such as the apparatus 100.

In FIG. 3A, a side elevation of the example arrangement, being a container like a paint can but with clear or transparent parts such as sides. The can may include a tube, or base, and lid configured so that the can is re-sealable, like a paint can or otherwise. The lid and a bottom of the can be metal, such as aluminum, or any suitable material.

In some embodiments the kit or package includes an item including a message positioned between each or at least two adjacent faux stones. The item may be scented, and include a small piece of recycled paper, for instance, and include messages like those described further below.

The container in various embodiments has an implement shaped and arranged at the container to give a desired appearance such as a natural appearance, like the grassy look shown. The implement can include a printed acetate liner, for instance, paint, sticker, etc. The implement can also include nature-shaped items, such as blades of grass, free to move, as shown in FIGS. 2A and 2B, for instance.

As shown in FIGS. 3A and 3B, a design, such as a partially-transparent or opaque rendering of grass can be provided on the side of the container, directly, or by way of an insert (FIG. 3B), for instance. This imaging can be reminiscent of that shown in FIGS. 2A and B, with both the imaging of FIGS. 2A, B and FIGS. 3A, B being reminiscent of natural rocks in nature.

FIG. 3B shows positioning of multiple faux stones being into a storage/display device with fragrant components. As also shown, in FIG. 3B, in some implementations, to provide fragrance to the faux-stone apparatus 100, a scented or scent-induced material (scented material) is provided. By way of example and not limitation, a drop of an oil—e.g., essential oil—or other fragrant material may be applied to a small piece of paper. In some implementations, the paper is recycled paper. In some implementations, any scent-releasing medium may be used. In some implementations, a scented piece of paper or other scented medium is placed atop a faux stone, under a faux stone, or between adjacent stones.

In a contemplated embodiment, the scented material is added to the faux-stone directly, during or after manufacture. The addition can be to the cover and/or the filler, such as by rendering a scented buckwheat filler.

The corresponding fragrance can be selected to induce a desired state or state change in the user, such as a relaxed state.

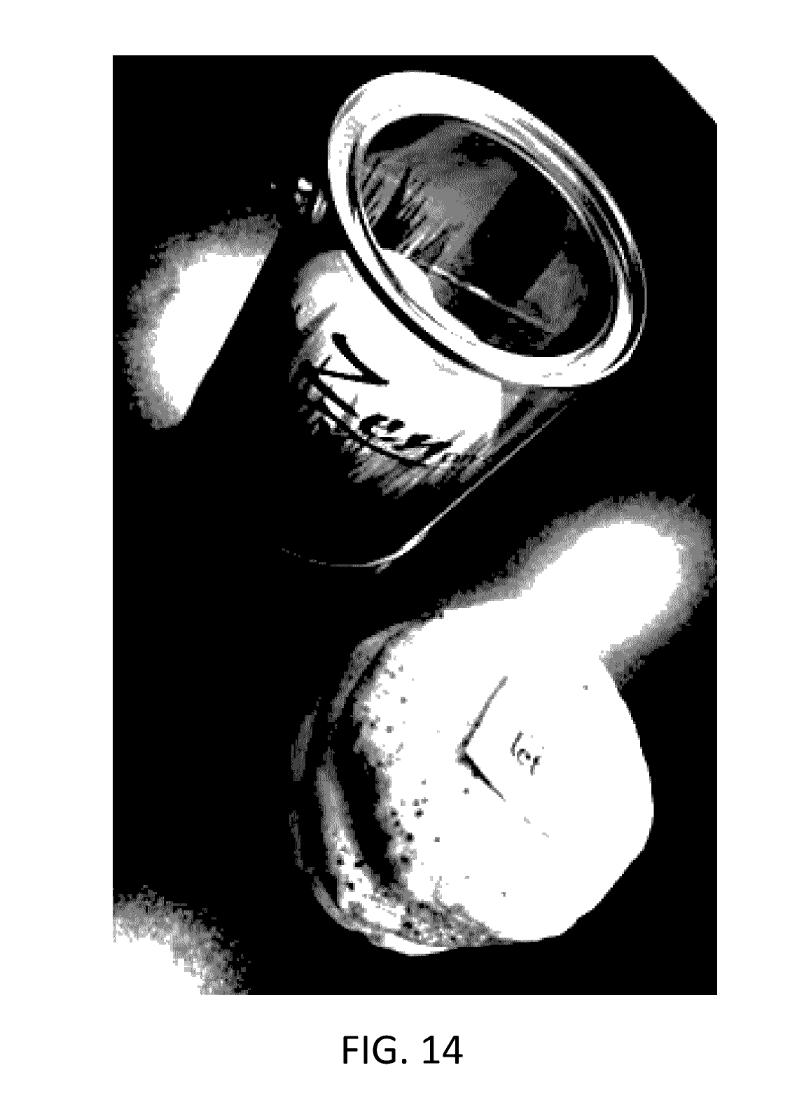

In some implementations, each paper will have one or more words and/or symbols imprinted thereon. In some implementations, the word or words are hand-stamped on the paper using stamp pad ink, for example, and can be colored as desired. In some implementations, the collection of words on the papers become a phrase and, in some implementations, the phrase becomes an offering of suggested intent, such as to “Go” on a first paper between a lowest stone and a second stone from the bottom), “It” on a second paper between the second stone and a third stone atop the second stone, and “Let” on a third paper atop the third stone (see e.g., FIGS. 14, 15, 17, 18), yields a message of “Let” “It” “Go”, a reference to relaxing, as a user moves the higher stones away in turn, such as when moving a stacked pile of the stones, whether from inside packaging or a stand-alone stack.

FIG. 3C shows a photo of faux stones positioned in the storage/display device. As shown in the example embodiment, the faux-stone apparatus can each have a single natural color such as grey of any shade, or more of a as shown in the photo, light brown, tan, beige, cream, or off-white, or any mixture or combination of these or other colors found actual rocks or stones in nature.

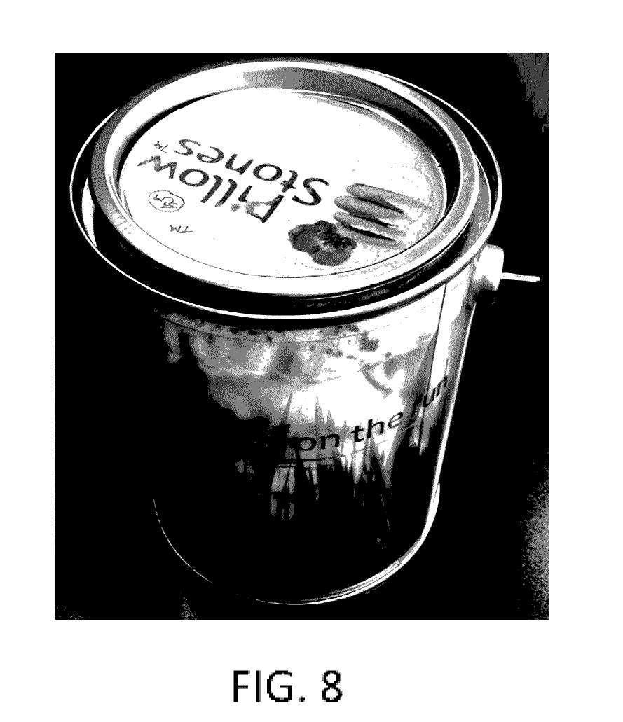

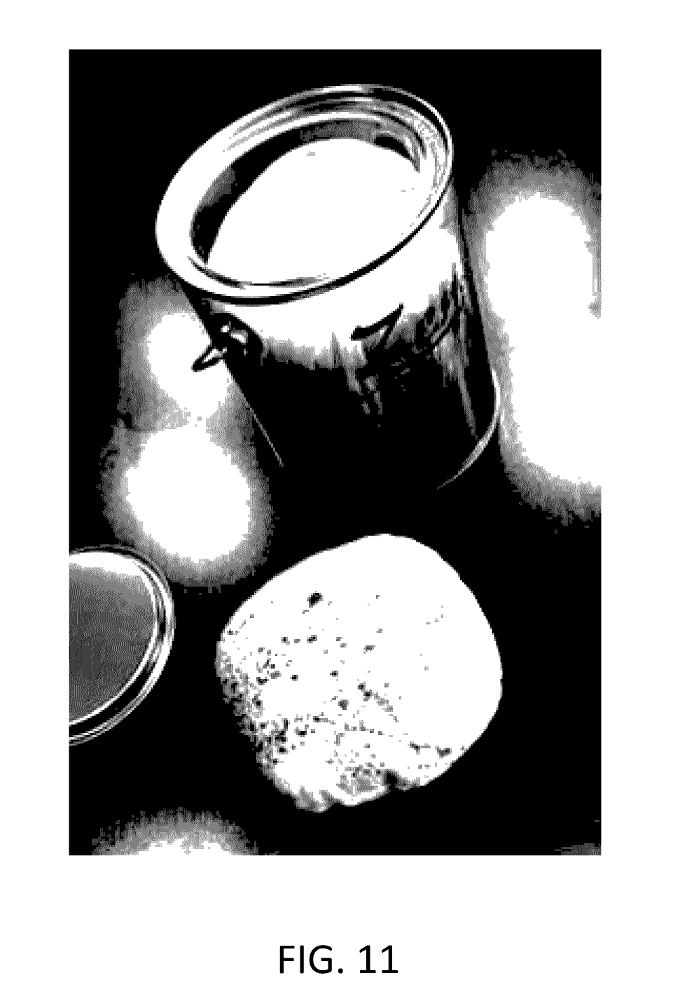

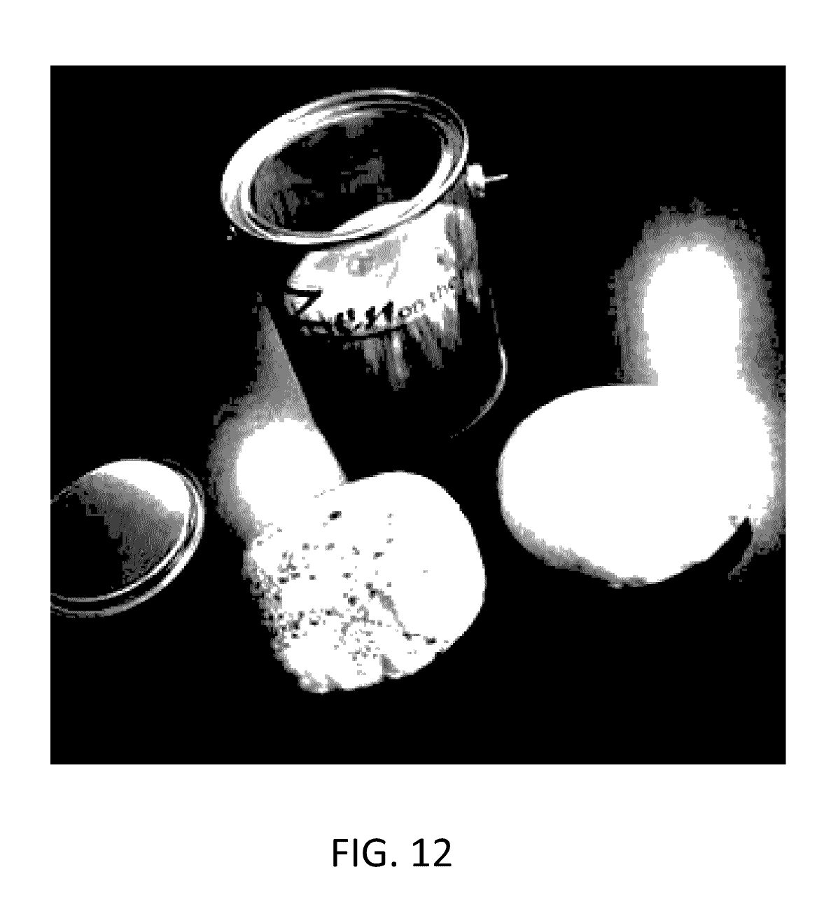

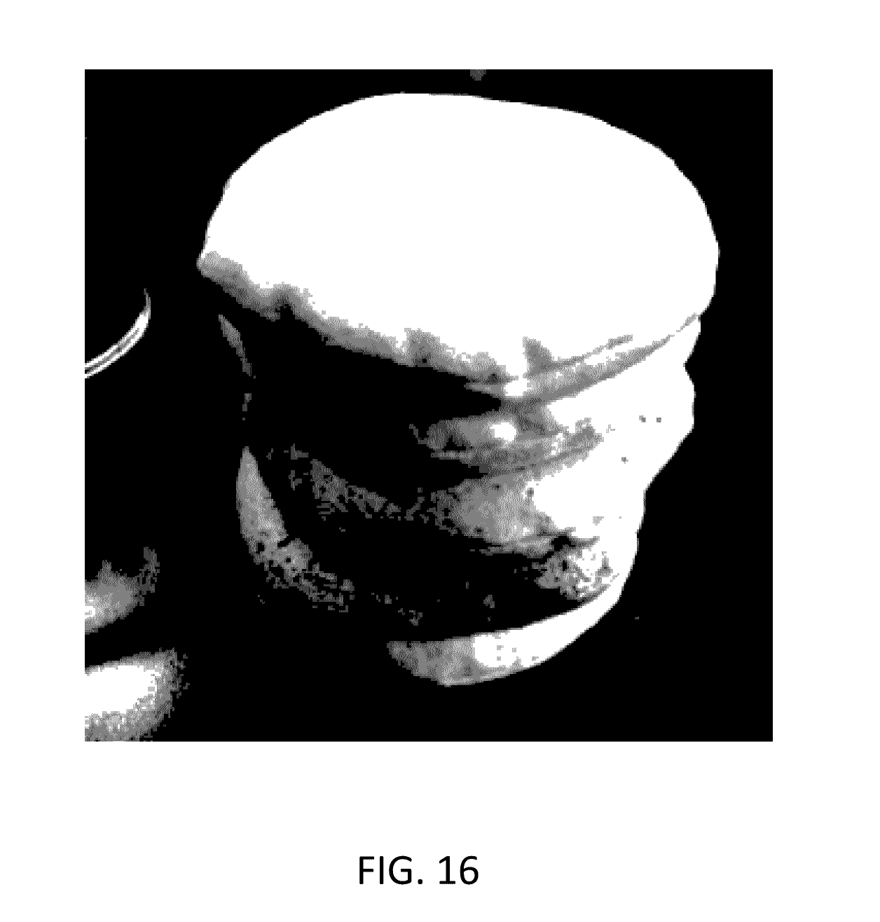

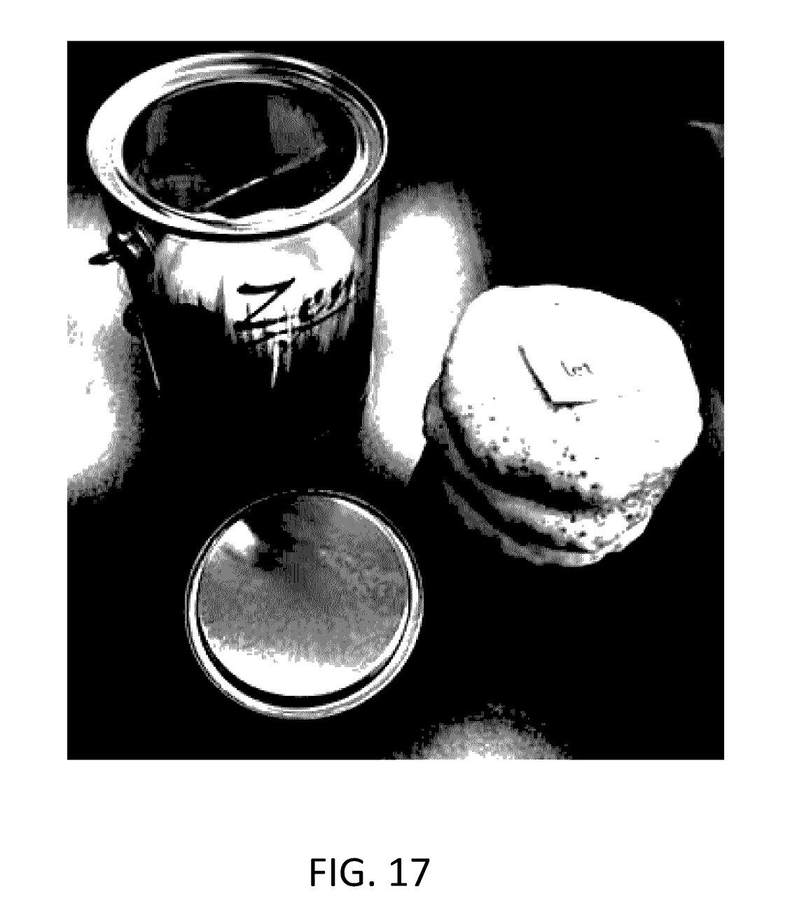

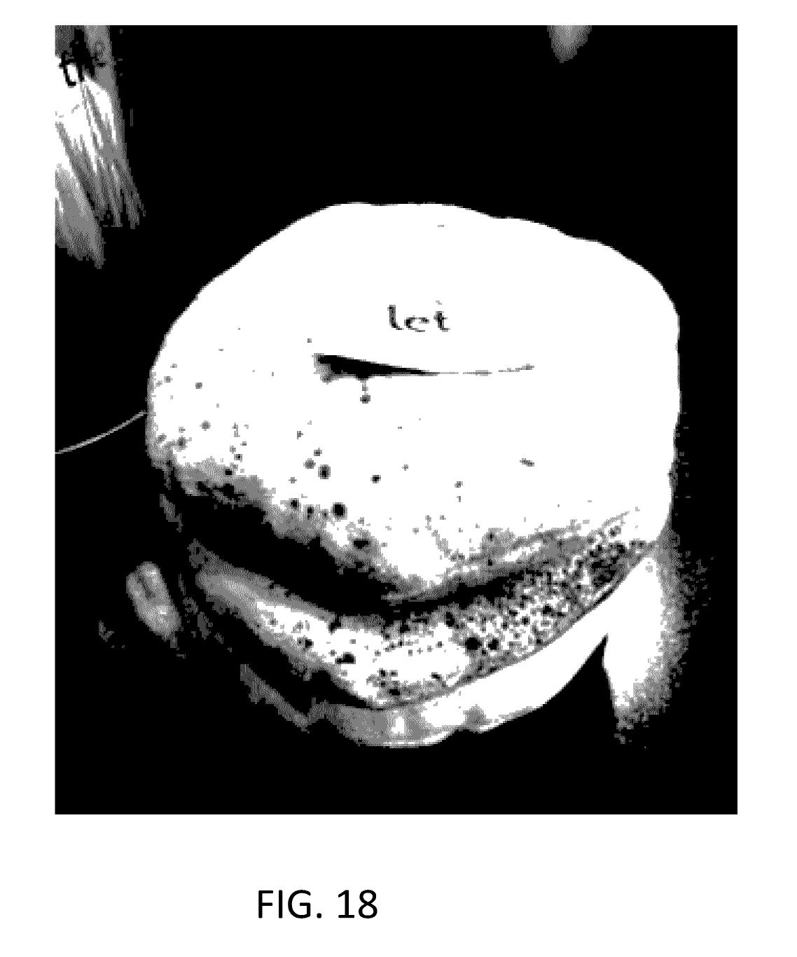

For examples of single-color finishes (e.g., light brown, tan, beige, cream, or off-white), see FIGS. 8-10 (second faux stone from the top in the container), FIG. 11 (top faux stone still in the container), FIGS. 12-15 (second faux stone on the table and the top faux stone still in the container), FIG. 16 (top two and bottom-most faux stones), and FIGS. 17, 18 (bottom-most faux stones).

For examples of speckled finishes, also see FIGS. 5-18.

FIGS. 3D, E show photos of the container from a perspective view standing up and on its side, respectively.

VII. Methods of Use—FIG. 4

Non-invasive body treatment methods of the present technology have various benefits. Benefits include promoting or stimulating circulation and relieving tension. The faux-stones of the present technology can be used in massage therapy.

The thermal-treatment processes include providing at least one faux-stone apparatus, and heating or cooling the faux-stone apparatus above or below room temperature, yielding a thermally-effected faux-stone apparatus.

The process further includes applying at least one thermally-effected faux-stone apparatus to any external portion of a body of the user. In various embodiments, the portion of a body of a user is pre-suggested by instructions made available to the user with the apparatus. The instructions can be provided by pamphlet in product packaging and/or on the packaging. See e.g., FIG. 3E showing example instructions on a bottom of a storage/display container.

Whether heated, cooled, or ambient (e.g., room temperature), a faux stone can be applied to any external part of the body, such as head, neck, chest, arms, hands, back, legs, and feet. In some implementations, faux stones are placed simultaneously against such parts of the body.

Each faux-stone creates a pressure effect on the user by touching the user. Even if slightly contacting the user, such as by being placed on a portion of the user body, the gravity of the apparatus affects the body. The user, or another such as a therapist or masseuse, can increase the pressure, such as by holding or pushing the apparatus into closer or stronger contact with the user.

These steps can be illustrated by a schematic flow diagram. The pre-suggested portion of the body corresponds to a chakra point of the body or a meridian point of the body. FIG. 4 illustrates example pre-suggested points on the body for application, which can referred to by any of a variety of terms, such as application points, contact points, hot points (for the warm or hot uses), cold points (for cool or cold uses), pressure points (for hot, cold, or ambient-temperature uses involving pressure by gravity and/or user or other—e.g., therapist), by way of examples.

As an example use according to the present disclosure, one or more faux-stone apparatus are warmed and/or cooled (e.g., some warmed, some cooled). In various implementations, the apparatus is warmed by heating them in a microwave, or by boiling water and using the steam from the water to heat the apparatus, or in any suitable manner

In implementations using a double boiler to heat faux-stone apparatus, for instance, the apparatus may be placed in the top cavity of the double boiler and then covered with lid for about 5 minutes to about 10 minutes. In some implementations, the boiling water is removed from the heat source before heating the apparatus.

In some embodiments, the apparatus is cooled by placing them in a freezer or cooling device. In some implementations, the apparatus is placed in a freezer for at least about 30 minutes. In some implementations, the apparatus may be placed in a freezer for less than about 30 minutes.

FIGS. 19 and 20 show three faux stone apparatus positioned on a user's back by way of example.

VIII. Additional Views and Embodiments—FIGS. 5-18

FIGS. 5-18 show various views of faux-stones inside and outside of the example display/storage container like that of FIG. 3.

As referenced above herein, the views show various embodiments including those in which faux stones are stacked, having a speckled finish, or a solid-colored finish (e.g., grey, cream, off-white, beige, light brown, etc.), those in which notes or symbols, such as on scented paper, are placed in the container and/or on, below, or between faux stones, and those in which the faux stones are positioned within or outside of the container.

IX. Removable Coverlet

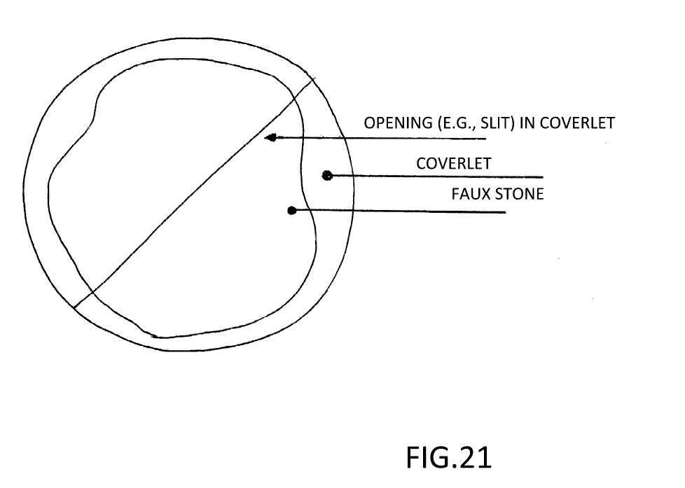

In some embodiments, a coverlet is available. By way of example, FIGS. 21-23 shows the coverlet, according to the present technology, by various views. The coverlet could also be referred to as a casing, a case, a pillow case, similar, or other.

The coverlet can be a part of a product kit including one or more faux stones, or provided separately. The coverlet is sized and shaped to fit around the cover, and in embodiments is machine-washable.

The coverlet is in some embodiments generally non-porous, such as to be water-proof, water-resistant, and/or easily cleaned between uses.

The coverlet is in some embodiments configured to cover the faux-stone apparatus while the apparatus is being used on a user. Benefits of this approach include receiving the benefit of using the apparatus while isolating the apparatus completely or substantially from certain elements, such as dust, dirt, skin, or oils in the use environment.

Keeping the apparatus clean can be especially useful in applications where a user, such as a salon owner or massage therapist would like to use the apparatus on more than one user over time.

FIG. 21 shows a see-through view of the coverlet in order to show the faux-stone inside.

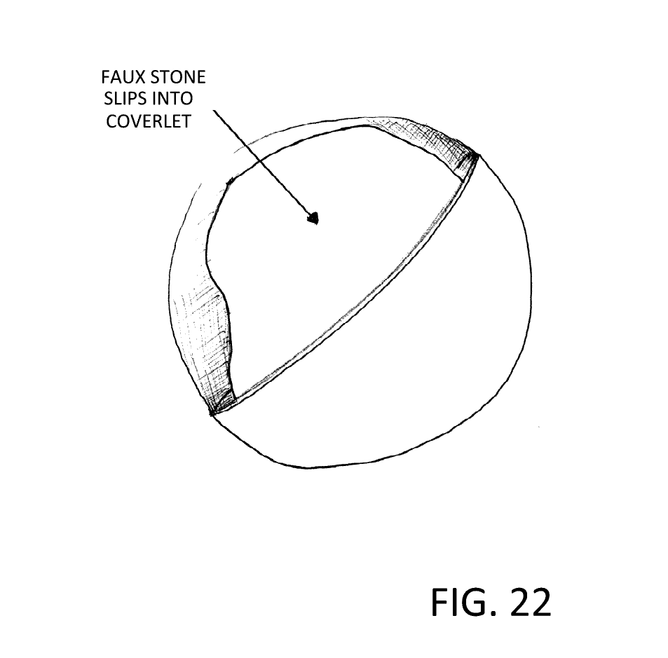

FIG. 22 shows a side view of the combination with the slit in the coverlet pulled open to expose the faux stone inside.

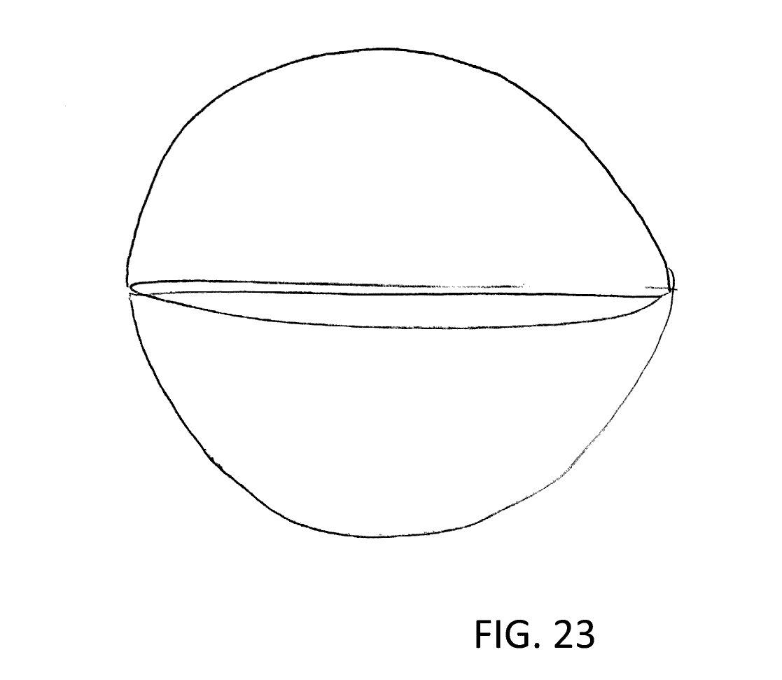

FIG. 23 shows a side view of the combination. The coverlet encapsulates the faux stone. In most embodiments, the coverlet is transparent, and so the faux stone inside would generally not be seen when surrounded by the coverlet.

X. Advantage and Benefits

Many of the advantages and benefits of the present technology including methods of use are described above.

Non-invasive body treatment methods of the present technology have various benefits. Benefits include promoting or stimulating circulation and relieving tension. The faux-stones of the present technology can be used in massage therapy.

Additional benefits include promoting meditation techniques.

The simple act of breathing, consciously, while using the product can enhance the user's experience, by offering a deeper reset and restoration as well as a greater sense of calm and clarity.

The faux-stone apparatus of the present disclosure are more portable than actual stones, being lighter and malleable/shapeable, fitting more easily in a user's purse or carry bag, for instance.

The faux-stone apparatus are relatively uncomplicated and inexpensive compared to most modern therapeutic devices including vibration devices, electric heat pads, those requiring expensive gels or other complex chemical combinations, those providing infrared heat therapy, and others requiring batteries or power cords.

The faux-stone apparatus is more ecofriendly than many products, such as those containing expensive gels or other complex chemical combinations, typically multiple man-made materials, such as plastic casings, electrical components, and/or and synthetic chemicals.

XI. Conclusion

Reference throughout this specification to “an embodiment” or “implementation” or words of similar import means that a particular described feature, structure, or characteristic is included in at least one embodiment of the present invention. Thus, the phrase “in an embodiment” or a phrase of similar import in various places throughout this specification does not necessarily refer to the same embodiment.

Modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

I claim:

1. A faux-stone apparatus, for providing a thermal-body treatment, comprising:
a natural granular filler comprising a multiplicity of natural granules, wherein:
(i) the granules are configured to, during operation of the faux-stone apparatus, receive heat input to the apparatus by conduction or radiation, retain heat received and, when positioned adjacent a portion of a user body after being heated, by radiation and conduction transfer heat to the user body to heat the body portion; and
(ii) the granules are configured to, during operation of the faux-stone apparatus, cool when exposed to a lower-temperature environment by transferring heat to the environment and, when the apparatus is positioned adjacent the user body after being cooled, receive heat from the user body thereby cooling the body portion; and
a natural cover that:
(a) is sized and shaped to fit in its entirety within an open hand of the user;
(b) is generally round, oval or irregularly shaped to look like a real stone; and
(c) fully encases and holds the granular filler.

2. The faux-stone apparatus of claim 1 wherein the cover is sized and shaped to fit mostly or entirely in a palm of the hand of the user;

3. The faux-stone apparatus of claim 1 wherein the natural granular filler comprises an organic material.

4. The faux-stone apparatus of claim 1 wherein the natural granular filler comprises at least one material selected from a group consisting of:
buckwheat;
raw buckwheat groats;
rice;
comprises millet;
comprises dried corn;
millet beans;
comprises flaxseed; and
cherry pits.

5. The faux-stone apparatus of claim 1 wherein the natural granular filler comprises pellets.

6. The faux-stone apparatus of claim 1 wherein the natural cover comprises flannel cotton or another type of cotton.

7. The faux-stone apparatus of claim 1 wherein the natural cover comprises flannel wool or another type of wool

8. The faux-stone apparatus of claim 1 wherein the natural cover, when encapsulating the natural granular filler to form the faux-stone apparatus, has a maximum width between about two inches and about five inches.

9. The faux-stone apparatus of claim 8 wherein the natural cover, when encapsulating the natural granular filler to form the faux-stone apparatus, has a maximum width between about two inches and about four inches.

10. The faux-stone apparatus of claim 9 wherein the natural cover has a maximum width between about two inches and about three inches.

11. The faux-stone apparatus of claim 1 further comprising a machine-washable coverlet sized and shaped to fit around the cover.

12. The faux-stone apparatus of claim 1 further comprising a generally non-porous coverlet sized and shaped to fit around the cover.

13. A thermal-treatment system, for providing a thermal body treatment, comprising a plurality of faux-stone apparatus, each faux-stone apparatus comprising:
a natural granular filler comprising a multiplicity of natural granules, wherein:
(I) the granules are configured to, during operation of the faux-stone apparatus, receive heat input to the apparatus by conduction or radiation, retain heat received and, when positioned adjacent a portion of a user body after being heated, by radiation and conduction transfer heat to the user body to heat the body portion; and
(II) the granules are configured to, during operation of the faux-stone apparatus, cool when exposed to a lower-temperature environment by transferring heat to the environment and, when the apparatus is positioned adjacent the user body after being cooled, receive heat from the user body thereby cooling the body portion; and
a natural cover that:
(A) is sized and shaped to fit in its entirety within an open hand of the user;
(B) is generally round, oval or irregularly shaped to look like a real stone; and
(C) fully encases and holds the granular filler.

14. The thermal-treatment system of claim 13 wherein the natural granular filler of each faux-stone apparatus comprises at least one material selected from a group consisting of:
buckwheat;
rice;
millet;
dried corn;
millet beans;
flaxseed; and
cherry pits.

15. The thermal-treatment system of claim 13 wherein the natural granular filler comprises at least one material selected from a group consisting of:
pellet-shaped material; and
an organic material.

16. The thermal-treatment system of claim 13 wherein the natural cover comprises at least one material selected from a group consisting of:
flannel cotton;
another type of cotton;
flannel wool; and
another type of wool.

17. The thermal-treatment system of claim 13 wherein each faux-stone apparatus comprises a coverlet that is sized and shaped to fit around the cover, and:
machine-washable; and/or
generally non-porous.

18. A thermal-treatment process comprising:
providing at least one faux-stone apparatus;
heating or cooling the faux-stone apparatus above or below room temperature, yielding a thermally-effected faux-stone apparatus; and
applying at least one thermally-effected faux-stone apparatus to a portion of a body of a user;
wherein:
each faux-stone apparatus comprises a natural granular filler comprising a multiplicity of natural granules, wherein:
(i) the granules are configured to, during operation of the faux-stone apparatus, receive heat input to the apparatus by conduction or radiation, retain heat received and, when, after being heated, the apparatus is positioned adjacent a portion of a user body, by radiation and conduction transfer the heat to the user body to heat the body portion; and
(ii) the granules are configured to, during operation of the faux-stone apparatus, cool when exposed to a lower-temperature environment by transferring heat to the environment and, when the apparatus is positioned adjacent the user body after being cooled, receive heat from the user body thereby cooling the body portion; and
faux-stone apparatus comprises a natural cover that:
(a) is sized and shaped to fit in its entirety within an open hand of the user;
(b) is generally round, oval or irregularly shaped to look like a real stone; and
(c) fully encases and holds the granular filler.

19. The thermal-treatment process of claim 18 further comprising:
providing a faux-stone usage instruction for use by the user or provider of the thermal-treatment process;
said portion of the body of the user is a pre-suggested user-body portion suggested by the faux-stone usage instruction. wherein the pre-suggested portion of applying at least one thermally-effected faux-stone apparatus to a portion of a body of a user;

20. The thermal-treatment process of claim 19 wherein the pre-suggested portion of the body corresponds to a chakra point of the body or a meridian point of the body.
